(19) United States
(12) Patent Application Publication
(10) Pub. No.: US 2026/0144660 A1
(43) Pub. Date: May 28, 2026

(54) KNEE BRACE

(71) Applicant: Guangdong Transcontinental Technology Co.,Ltd., Guangzhou City (CN)

(72) Inventors: HONG ZHOU, Guangzhou City (CN); ZHENTONG YANG, Guangzhou City (CN); ZHIXIN PENG, Guangzhou City (CN)

(21) Appl. No.: 18/966,770

(22) Filed: Dec. 3, 2024

(30) Foreign Application Priority Data
Nov. 27, 2024 (CN) 202422910696.3

(51) Int. Cl. A61F 5/01

(52) U.S. Cl. CPC A61F 5/0123

(57) ABSTRACT

A knee brace includes a knee brace main body and a first reinforcing band part. A side wall of the knee brace main body surrounds and forms a wrapping space for wrapping a knee. A first end of the first reinforcing band part is connected to a first part of the knee brace main body. A second end of the first reinforcing band part is detachably connected to a second part of the knee brace main body. The first reinforcing band part is used for shrinking the wrapping space. A user can first wrap the knee brace main body around the knee, then connect the first end of the first reinforcing band part to the first part of the knee brace main body, and connect the second end of the first reinforcing band part to the second part of the knee brace main body, thereby supporting and protecting the knee.

### CROSS-REFERENCE TO RELATED APPLICATIONS

The application claims priority of Chinese patent application CN2024229106963, filed on Nov. 27, 2024, which is incorporated herein by reference in its entireties.

### TECHNICAL FIELD

The present invention relates to the field of protective gear, particularly to a knee brace.

### BACKGROUND ART

The knee joint is an important load-bearing joint in the human body. During exercise, labor, or daily life, the knee joint bears enormous pressure. Generally, knee braces can be used to protect the knee joint, including preventing knee damage, relieving pressure on the knee, keeping warm, preventing arthritis, and accelerating the rehabilitation process. However, existing knee braces on the market often fail to tighten the knee well and are prone to loosening, resulting in a poor protection effect of the knee braces on the knee. Therefore, there is an urgent need on the market to provide a knee brace with high tightening force and good knee protection effect to improve the user experience.

### SUMMARY

In order to overcome the shortcomings of the prior art, a knee brace is provided in the present invention. The knee brace includes:
a knee brace main body, wherein a side wall of the knee brace main body surrounds and forms a wrapping space for wrapping a knee; and
a first reinforcing band part, wherein a first end of the first reinforcing band part is connected to a first part of the knee brace main body, a second end of the first reinforcing band part is detachably connected to a second part of the knee brace main body, and the first reinforcing band part is used for shrinking the wrapping space.

As an improvement of the present invention, the knee brace main body includes a first connecting area and a third connecting part. The first end of the first reinforcing band part is connected to the third connecting part, and the second end of the first reinforcing band part is detachably connected to the first connecting area. The first reinforcing band part is used for tightening the first connecting area in a direction towards the third connecting part, so that the first reinforcing band part shrinks the wrapping space.

As an improvement of the present invention, the first connecting area is provided with a plurality of first connecting portions, and the first reinforcing band part is provided with first connecting fitting portions. The first connecting fitting portion is selectively connected to one of the plurality of first connecting portions to adjust a size of the wrapping space.

As an improvement of the present invention, the knee brace further includes a second reinforcing band part, and the knee brace main body further includes a second connecting area. A first end of the second reinforcing band part is connected to the third connecting part, and a second end of the second reinforcing band part is detachably connected to the second connecting area. The second reinforcing band part is used for tightening the second connecting area in the direction towards the third connecting part, so that the second reinforcing band part shrinks the wrapping space.

As an improvement of the present invention, the second connecting area is provided with a plurality of second connecting portions, and the second reinforcing band part is provided with second connecting fitting portions. The second connecting fitting portion is selectively connected to one of the plurality of second connecting portions to adjust a size of the wrapping space.

As an improvement of the present invention, the knee brace further includes a third reinforcing band part, and the knee brace main body further includes a third connecting area. A first end of the third reinforcing band part is connected to the third connecting part, and a second end of the third reinforcing band part is detachably connected to the third connecting area. The third reinforcing band part is used for tightening the third connecting area in the direction towards the third connecting part, so that the third reinforcing band part shrinks the wrapping space.

As an improvement of the present invention, the third connecting area is provided with a plurality of third connecting portions, and the third reinforcing band part is provided with third connecting fitting portions. The third connecting fitting portion is selectively connected to one of the plurality of third connecting portions to adjust a size of the wrapping space.

As an improvement of the present invention, the knee brace further includes a fourth reinforcing band part, and the knee brace main body further includes a fourth connecting area. A first end of the fourth reinforcing band part is connected to the third connecting part, and a second end of the fourth reinforcing band part is detachably connected to the fourth connecting area. The fourth reinforcing band part is used for tightening the fourth connecting area in the direction towards the third connecting part, so that the fourth reinforcing band part shrinks the wrapping space.

As an improvement of the present invention, the fourth connecting area is provided with a plurality of fourth connecting portions, and the fourth reinforcing band part is provided with fourth connecting fitting portions. The fourth connecting fitting portion is selectively connected to one of the plurality of fourth connecting portions to adjust a size of the wrapping space.

As an improvement of the present invention, an extension direction of the first reinforcing band part is different from an extension direction of the second reinforcing band part.

As an improvement of the present invention, the third reinforcing band part and the first reinforcing band part extend in opposite directions to each other. The fourth reinforcing band part and the second reinforcing band part extend in opposite directions to each other.

As an improvement of the present invention, the first reinforcing band part, the second reinforcing band part, the third reinforcing band part, and the fourth reinforcing band part are distributed in a cross shape.

As an improvement of the present invention, the first connecting portion is a first hook-and-loop fitting band, and the first connecting fitting portion is a first hook-and-loop band. The second connecting portion is a second hook-and-loop fitting band, and the second connecting fitting portion is a second hook-and-loop band. The third connecting portion is a third hook-and-loop fitting band, and the third connecting fitting portion is a third hook-and-loop band. The fourth connecting portion is a fourth hook-and-loop fitting band, and the fourth connecting fitting portion is a fourth hook-and-loop band.

As an improvement of the present invention, one side of the knee brace main body is equipped with a first connecting part, and an opposite side of the knee brace main body is equipped with a second connecting part. The first connecting part is connected to the second connecting part, so that the side wall of the knee brace main body surrounds and forms the wrapping space for wrapping the knee.

As an improvement of the present invention, the knee brace further includes a supporting part. The first connecting part is positioned on a left side of the knee brace main body. The second connecting part is positioned on a right side of the knee brace main body. The supporting part extends from an upper side of the knee brace main body to a lower side of the knee brace main body.

As an improvement of the present invention, the supporting part includes a plurality of elastic first supporting steel ribs and a plurality of elastic second supporting steel ribs. The plurality of first supporting steel ribs are arranged at intervals on the left side of the knee brace main body, and the plurality of second supporting steel ribs are arranged at intervals on the right side of the knee brace main body.

As an improvement of the present invention, the first supporting steel rib is formed by a plurality of first steel rings being sequentially connected. The second supporting steel rib is formed by a plurality of second steel rings being sequentially connected.

As an improvement of the present invention, a plurality of first installation chambers are defined in the left side of the knee brace main body, and a plurality of second installation chambers are defined in the right side of the knee brace main body. The plurality of first installation chambers are separated from each other, and the plurality of second installation chambers are separated from each other. The first supporting steel rib is positioned inside the first installation chamber, and the second supporting steel rib is positioned inside the second installation chamber.

As an improvement of the present invention, the first connecting part includes a protruding first connecting band and a protruding second connecting band. The second connecting part includes a protruding third connecting band and a protruding fourth connecting band. The first connecting band is detachably connected to the third connecting band, and the second connecting band is detachably connected to the fourth connecting band. A first avoidance gap is defined between the first connecting band and the second connecting band. A second avoidance gap is defined between the third connecting band and the fourth connecting band. A length of the first connecting band is greater than a length of the second connecting band, and a length of the third connecting band is greater than a length of the fourth connecting band.

As an improvement of the present invention, the knee brace further includes a knee brace cover. The knee brace cover is detachably connected to the knee brace main body.

As an improvement of the present invention, a hardness of the knee brace cover is greater than a hardness of the knee brace main body. The knee brace cover includes a plastic cover and a flexible layer. The plastic cover is connected to the flexible layer, and the flexible layer is detachably connected to the knee brace main body. The third connecting part is positioned in a center of the knee brace main body. The knee brace cover detachably covers the third connecting part. The third connecting part is in an annular shape, and the third connecting part protrudes from a surface of the knee brace main body. An avoidance through hole is defined in a middle portion of the third connecting part.

As an improvement of the present invention, the flexible layer is a flexible cotton layer.

As an improvement of the present invention, the knee brace further includes a knee brace cover. The first end of the first reinforcing band part is detachably connected to the third connecting part through the knee brace cover, and the second end of the first reinforcing band part is detachably connected to the first connecting area. The first reinforcing band part is used for tightening the first connecting area in the direction towards the third connecting part and the knee brace cover, so that the first reinforcing band part shrinks the wrapping space.

Beneficial effects of the present invention are as follows. The knee brace is provided in the present invention. The knee brace includes the knee brace main body and the first reinforcing band part. The side wall of the knee brace main body surrounds and forms the wrapping space for wrapping the knee. The first end of the first reinforcing band part is connected to the first part of the knee brace main body, and the second end of the first reinforcing band part is detachably connected to the second part of the knee brace main body. The first reinforcing band part is used for shrinking the wrapping space. Therefore, a user can first wrap the knee brace main body around the knee, so that the wrapping space wraps and fixes the knee, allowing the knee brace main body to provide stable support and protection for the knee. Afterwards, the first end of the first reinforcing band part is connected to the first part of the knee brace main body, and the second end of the first reinforcing band part is connected to the second part of the knee brace main body, so that the first reinforcing band part tightens the second part of the knee brace main body in the direction towards the first part of the knee brace main body, further shrinking the wrapping space, allowing the knee brace main body to further tighten the knee, allowing the knee brace main body to provide more stable support and protection to the knee, thereby improving a knee protection effect of the knee brace main body. Moreover, due to the detachable connection between the second end of the first reinforcing band part and the second part of the knee brace main body, it is convenient for the user to quickly put on and take off the knee brace.

### BRIEF DESCRIPTION OF THE DRAWINGS

Implementations of the present disclosure will now be described, by way of embodiment, with reference to the attached figures. It should be understood, the drawings are shown for illustrative purpose only, for ordinary person skilled in the art, other drawings obtained from these drawings without paying creative labor by an ordinary person skilled in the art should be within scope of the present disclosure.

FIG. 1 is a schematic diagram of an overall structure of the present invention.

FIG. 2 is another schematic diagram of an overall structure of the present invention.

FIG. 3 is another schematic diagram of an overall structure of the present invention.

FIG. 4 is an exploded view of the present invention.

FIG. 5 is another exploded view of the present invention.

FIG. 6 is a sectional view of a knee brace main body and a supporting part.

FIG. 7 is an enlarged view of area A in FIG. 6.

FIG. 8 is another exploded view of the present invention.

### DETAILED DESCRIPTION OF THE EMBODIMENTS

It will be appreciated that for simplicity and clarity of illustration, where appropriate, reference numerals have been repeated among the different figures to indicate corresponding or analogous elements. In addition, numerous specific details are set forth in order to provide a thorough understanding of the exemplary embodiments described herein. However, it will be understood by those of ordinary skill in the art that the exemplary embodiments described herein may be practiced without these specific details. In other instances, methods, procedures, and components have not been described in detail so as not to obscure the related relevant feature being described. Also, the description is not to be considered as limiting the scope of the exemplary embodiments described herein. The drawings are not necessarily to scale and the proportions of certain parts may be exaggerated to better illustrate details and features of the present disclosure.

The term “comprising” when utilized, means “including, but not necessarily limited to”; it specifically indicates open-ended inclusion or membership in the so-described combination, group, series, and the like. The disclosure is illustrated by way of example and not by way of limitation in the figures of the accompanying drawings in which like references indicate similar elements. It should be noted that references to “an” or “one” embodiment in this disclosure are not necessarily to the same embodiment, and such references can mean “at least one”. In addition, the terms “first” and “second” are used for descriptive purposes only and cannot be understood as indicating or implying relative importance or implying the number of indicated technical features. Thus, the features defined as “first” and “second” may explicitly or implicitly include one or more of the features. In the description of embodiments of the application, “a plurality of” means two or more, unless otherwise specifically defined.

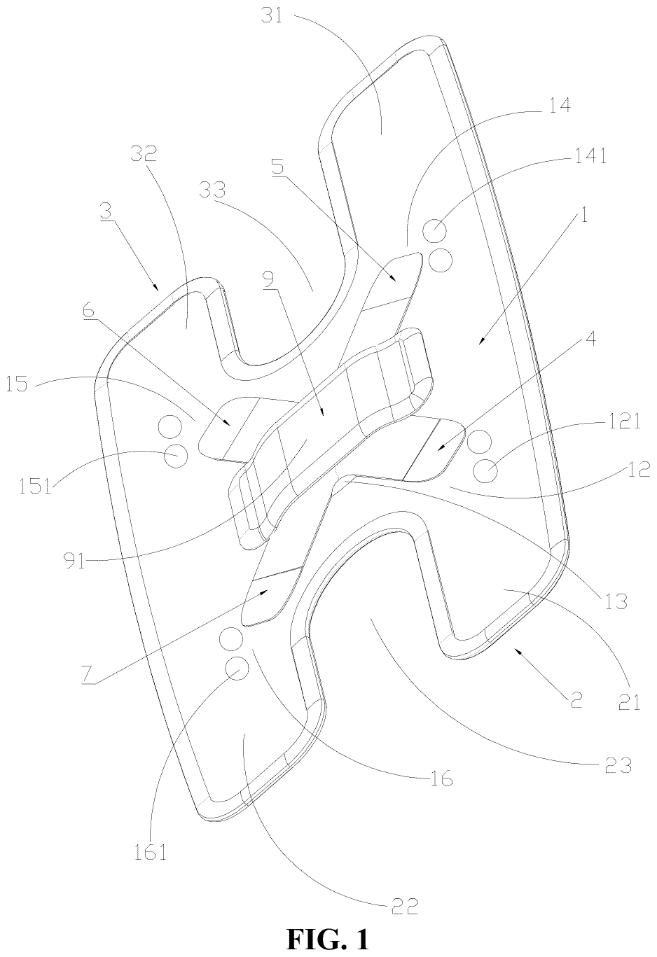

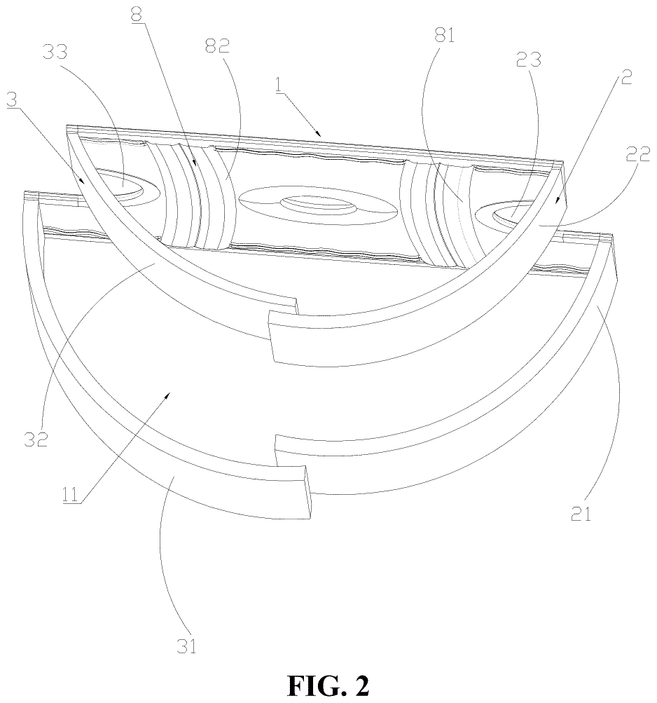

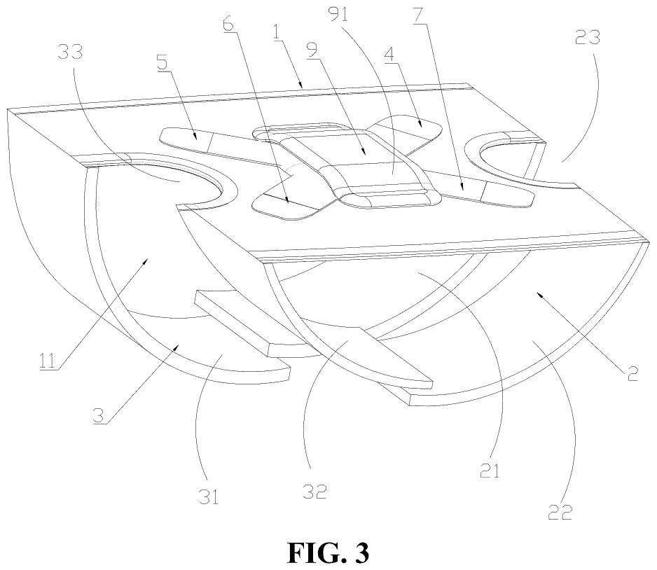

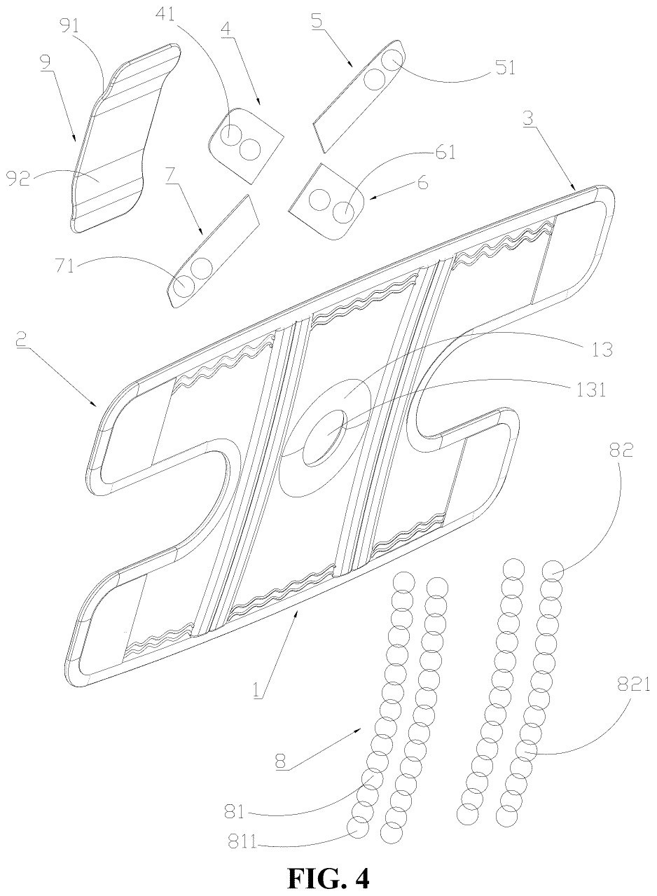

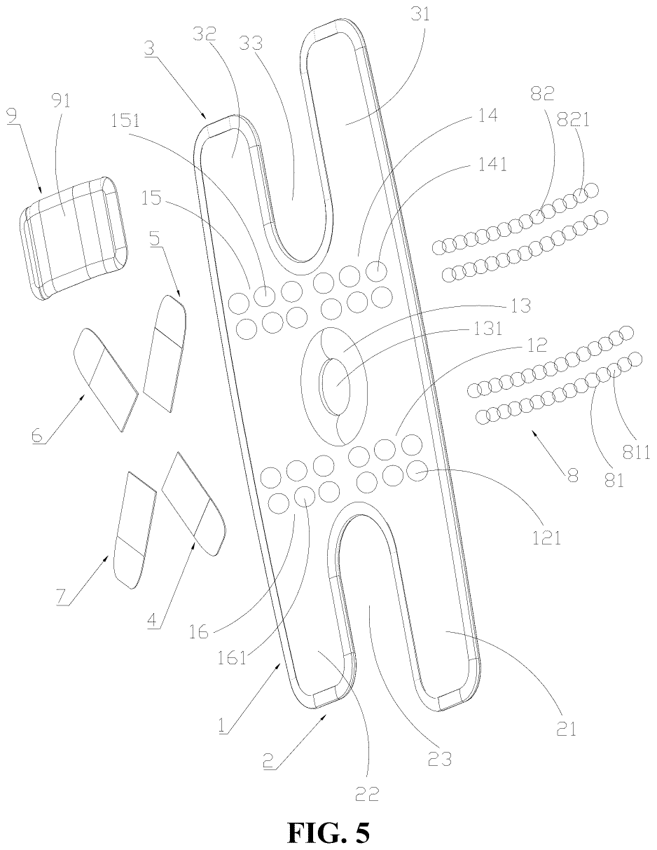

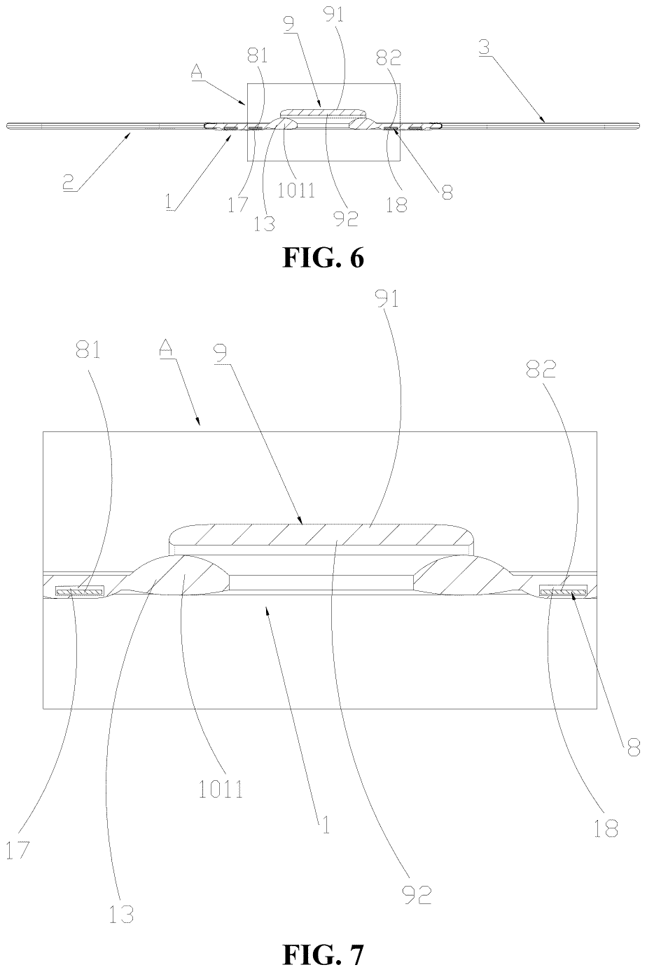

Referring to FIGS. 1-8, a knee brace includes:
a knee brace main body 1, wherein a side wall of the knee brace main body 1 surrounds and forms a wrapping space 11 for wrapping a knee; and
a first reinforcing band part 4, wherein a first end of the first reinforcing band part 4 is connected to a first part of the knee brace main body 1, a second end of the first reinforcing band part 4 is detachably connected to a second part of the knee brace main body 1, and the first reinforcing band part 4 is used for shrinking the wrapping space 11.

Through the above structure, the knee brace includes the knee brace main body 1 and the first reinforcing band part 4. The side wall of the knee brace main body 1 surrounds and forms the wrapping space 11 for wrapping the knee. The first end of the first reinforcing band part 4 is connected to the first part of the knee brace main body 1, and the second end of the first reinforcing band part 4 is detachably connected to the second part of the knee brace main body 1. The first reinforcing band part 4 is used for shrinking the wrapping space 11. Therefore, a user can first wrap the knee brace main body 1 around the knee, so that the wrapping space 11 wraps and fixes the knee, allowing the knee brace main body 1 to provide stable support and protection for the knee. Afterwards, the first end of the first reinforcing band part 4 is connected to the first part of the knee brace main body 1, and the second end of the first reinforcing band part 4 is connected to the second part of the knee brace main body 1, so that the first reinforcing band part 4 tightens the second part of the knee brace main body 1 in a direction towards the first part of the knee brace main body 1, further shrinking the wrapping space 11, allowing the knee brace main body 1 to further tighten the knee, allowing the knee brace main body 1 to provide more stable support and protection to the knee, thereby improving a knee protection effect of the knee brace main body 1. Moreover, due to the detachable connection between the second end of the first reinforcing band part 4 and the second part of the knee brace main body 1, it is convenient for the user to quickly put on and take off the knee brace.

In this embodiment, the knee brace main body 1 includes a first connecting area 12 and a third connecting part 13. The first end of the first reinforcing band part 4 is connected to the third connecting part 13, and the second end of the first reinforcing band part 4 is detachably connected to the first connecting area 12. The first reinforcing band part 4 is used for tightening the first connecting area 12 in a direction towards the third connecting part 13, so that the first reinforcing band part 4 shrinks the wrapping space 11. Through the above structure, the first reinforcing band part 4 tightens the first connecting area 12 in the direction towards the third connecting part 13, so that the first reinforcing band part 4 shrinks the wrapping space 11, allowing the knee brace main body 1 to further tighten the knee, and allowing the knee brace main body 1 to provide more stable support and protection to the knee, thereby improving the knee protection effect of the knee brace main body 1. When the user needs to wear or remove the knee brace, the second end of the first reinforcing band part 4 can be separated from the first connecting area 12 to expand the wrapping space 11, making it easier for the user to quickly put on and take off the knee brace.

In this embodiment, the first connecting area 12 is provided with a plurality of first connecting portions 121, and the first reinforcing band part 4 is provided with first connecting fitting portions 41. The first connecting fitting portion 41 is selectively connected to one of the plurality of first connecting portions 121 to adjust a size of the wrapping space 11. Through the above structure, since the first connecting fitting portion 41 is selectively connected to one of the plurality of first connecting portions 121, it is convenient for the user to adjust the size of the wrapping space 11, adjust a contraction amount of the wrapping space 11, and adjust tightness of the knee brace main body 1 wrapping the knee to meet actual usage needs of the user in different scenarios.

In this embodiment, the knee brace further includes a second reinforcing band part 5, and the knee brace main body 1 further includes a second connecting area 14. A first end of the second reinforcing band part 5 is connected to the third connecting part 13, and a second end of the second reinforcing band part 5 is detachably connected to the second connecting area 14. The second reinforcing band part 5 is used for tightening the second connecting area 14 in the direction towards the third connecting part 13, so that the second reinforcing band part 5 shrinks the wrapping space 11. The second connecting area 14 is provided with a plurality of second connecting portions 141, and the second reinforcing band part 5 is provided with second connecting fitting portions 51. The second connecting fitting portion 51 is selectively connected to one of the plurality of second connecting portions 141 to adjust a size of the wrapping space 11. Through the above structure, the first reinforcing band part 4 and the second reinforcing band part 5 jointly shrink the wrapping space 11, so that the knee brace main body 1 provides more stable support and protection to the knee, thereby improving the knee protection effect of the knee brace main body 1. Moreover, an extension direction of the first reinforcing band part 4 is different from an extension direction of the second reinforcing band part 5, so that the first reinforcing band part 4 and the second reinforcing band part 5 can jointly contract the wrapping space 11 from different directions, making the knee brace main body 1 contract more evenly and providing more uniform support to the knee brace main body 1.

In this embodiment, the knee brace further includes a third reinforcing band part 6, and the knee brace main body 1 further includes a third connecting area 15. A first end of the third reinforcing band part 6 is connected to the third connecting part 13, and a second end of the third reinforcing band part is detachably connected to the third connecting area 15. The third reinforcing band part 6 is used for tightening the third connecting area 15 in the direction towards the third connecting part 13, so that the third reinforcing band part 6 shrinks the wrapping space 11. The third connecting area 15 is provided with a plurality of third connecting portions 151, and the third reinforcing band part 6 is provided with third connecting fitting portions 61. The third connecting fitting portion 61 is selectively connected to one of the plurality of third connecting portions 151 to adjust a size of the wrapping space 11. Specifically, the knee brace further includes a fourth reinforcing band part 7, and the knee brace main body 1 further includes a fourth connecting area 16. A first end of the fourth reinforcing band part 7 is connected to the third connecting part 13, and a second end of the fourth reinforcing band part 7 is detachably connected to the fourth connecting area 16. The fourth reinforcing band part 7 is used for tightening the fourth connecting area 16 in the direction towards the third connecting part 13, so that the fourth reinforcing band part 7 shrinks the wrapping space 11. The fourth connecting area 16 is provided with a plurality of fourth connecting portions 161, and the fourth reinforcing band part 7 is provided with fourth connecting fitting portions 71. The fourth connecting fitting portion 71 is selectively connected to one of the plurality of fourth connecting portions 161 to adjust a size of the wrapping space 11. Through the above structure, the first reinforcing band part 4, the second reinforcing band part 5, the third reinforcing band part 6, and the fourth reinforcing band part 7 collectively contract the wrapping space 11, so that the knee brace main body 1 provides more stable support and protection to the knee, thereby improving the knee protection effect of the knee brace main body 1.

In this embodiment, the third reinforcing band part 6 and the first reinforcing band part 4 extend in opposite directions to each other. The fourth reinforcing band part 7 and the second reinforcing band part 5 extend in opposite directions to each other. The first reinforcing band part 4, the second reinforcing band part 5, the third reinforcing band part 6, and the fourth reinforcing band part 7 are distributed in a cross shape. Through the above structure, the first reinforcing band part 4, the second reinforcing band part 5, the third reinforcing band part 6, and the fourth reinforcing band part 7 can collectively contract the wrapping space 11 from four different directions, making the knee brace main body 1 contract more evenly and providing more uniform support to the knee brace main body 1. Moreover, since the first reinforcing band part 4, the second reinforcing band part 5, the third reinforcing band part 6, and the fourth reinforcing band part 7 are distributed in a cross shape, the knee brace main body 1 can simultaneously contract towards an intersection point, i.e., the third connecting part 13, allowing the knee brace main body 1 to contract more evenly and provide more uniform support for the knee, thus protecting the knee more effectively. The first connecting area 12 is positioned on an upper side of the knee brace main body 1, the second connecting area 14 is positioned on a right side of the first connecting area 12, the third connecting area 15 is positioned below the first connecting area 12, and the fourth connecting area 16 is positioned below the second connecting area 14.

In this embodiment, the first connecting portion 121 is a first hook-and-loop fitting band, and the first connecting fitting portion 41 is a first hook-and-loop band. The second connecting portion 141 is a second hook-and-loop fitting band, and the second connecting fitting portion 51 is a second hook-and-loop band. The third connecting portion 151 is a third hook-and-loop fitting band, and the third connecting fitting portion 61 is a third hook-and-loop band. The fourth connecting portion 161 is a fourth hook-and-loop fitting band, and the fourth connecting fitting portion 71 is a fourth hook-and-loop band. Through the above structure, the detachable connection between the first reinforcing band part 4, the second reinforcing band part 5, the third reinforcing band part 6, and the fourth reinforcing band part 7 and the knee brace main body 1 is effectively achieved, and it is convenient for the user to quickly and accurately adjust the size of the wrapping space 11 from various directions. The first hook-and-loop band, the second hook-and-loop band, the third hook-and-loop band, and the fourth hook-and-loop band are all hook-and-loop bands on hook-and-loop fasteners. The first hook-and-loop fitting band, the second hook-and-loop fitting band, the third hook-and-loop fitting band, and the fourth hook-and-loop fitting band are all hook-and-loop fitting bands on cotton cloth.

In this embodiment, one side of the knee brace main body 1 is equipped with a first connecting part 2, and an opposite side of the knee brace main body 1 is equipped with a second connecting part 3. The first connecting part 2 is connected to the second connecting part 3, so that the side wall of the knee brace main body 1 surrounds and forms the wrapping space 11 for wrapping the knee. Through the above structure, the user can first connect the first connecting part 2 with the second connecting part 3 to wrap the knee brace main body 1 around the knee, so that the wrapping space 11 can wrap and fix the knee brace, allowing the knee brace main body 1 to provide the knee with stable support and protection. Afterwards, the first end of the first reinforcing band part 4 is connected to the first part of the knee brace main body 1, and the second end of the first reinforcing band part 4 is connected to the second part of the knee brace main body 1, so that the first reinforcing band part 4 tightens the second part of the knee brace main body 1 in the direction towards the first part of the knee brace main body 1, further shrinking the wrapping space 11, allowing the knee brace main body 1 to further tighten the knee to provide more stable support and protection to the knee, thereby improving the knee protection effect of the knee brace main body 1. Moreover, due to the detachable connection between the second end of the first reinforcing band part 4 and the second part of the knee brace main body 1, it is convenient for the user to quickly put on and take off the knee brace.

In this embodiment, the knee brace further includes a supporting part 8. The first connecting part 2 is positioned on a left side of the knee brace main body 1. The second connecting part 3 is positioned on a right side of the knee brace main body 1. The supporting part 8 extends from an upper side of the knee brace main body 1 to a lower side of the knee brace main body 1. The supporting part 8 includes a plurality of elastic first supporting steel ribs 81 and a plurality of elastic second supporting steel ribs 82. The plurality of first supporting steel ribs 81 are arranged at intervals on the left side of the knee brace main body 1, and the plurality of second supporting steel ribs 82 are arranged at intervals on the right side of the knee brace main body 1. Specifically, the first supporting steel rib 81 is formed by a plurality of first steel rings 811 being sequentially connected. The second supporting steel rib 82 is formed by a plurality of second steel rings 821 being sequentially connected. Furthermore, a plurality of first installation chambers 17 are defined in the left side of the knee brace main body 1, and a plurality of second installation chambers 18 are defined in the right side of the knee brace main body 1. The plurality of first installation chambers 17 are separated from each other, and the plurality of second installation chambers 18 are separated from each other. The first supporting steel rib 81 is positioned inside the first installation chamber 17, and the second supporting steel rib 82 is positioned inside the second installation chamber 18. Through the above structure, the first supporting steel rib 81 and the second supporting steel rib 82 can support a left side and a right side of the knee, making the knee more stable, and providing support to the left side and the right side of the knee to protect the knee. Moreover, the elastic first supporting steel rib 81 and the elastic second supporting steel rib 82 can better fit a curve of the knee. Furthermore, the plurality of first installation chambers 17 are defined in the left side of the knee brace main body 1, the plurality of second installation chambers 18 are defined in the right side of the knee brace main body 1, the plurality of first installation chambers 17 are separated from each other, the plurality of second installation chambers 18 are separated from each other, the first supporting steel rib 81 is positioned in the first installation chamber 17, and the second supporting steel rib 82 is positioned in the second installation chamber 18, so that each first supporting steel rib 81 can be stably placed in the first installation chamber 17, and each second supporting steel rib 82 can be stably placed in the second installation chamber 18, preventing positions of the first supporting steel rib 81 and the second supporting steel rib 82 from shifting, and allowing the first supporting steel rib 81 and the second supporting steel rib 82 to more stably support the left side and the right side of the knee. A total number of the first supporting steel ribs 81 is two, and a total number of the second supporting steel ribs 82 is two.

In this embodiment, the first connecting part 2 includes a protruding first connecting band 21 and a protruding second connecting band 22. The second connecting part 3 includes a protruding third connecting band and a protruding fourth connecting band 32. The first connecting band 21 is detachably connected to the third connecting band 31, and the second connecting band 22 is detachably connected to the fourth connecting band 32. A first avoidance gap 23 is defined between the first connecting band 21 and the second connecting band 22. A second avoidance gap 33 is defined between the third connecting band 31 and the fourth connecting band 32. A length of the first connecting band 21 is greater than a length of the second connecting band 22, and a length of the third connecting band 31 is greater than a length of the fourth connecting band 32. Through the above structure, the first avoidance gap 23 and the second avoidance gap 33 can avoid a popliteal fossa, allowing the knee brace main body 1 to wrap around the knee without affecting blood circulation in the popliteal fossa, and keeping blood vessels in a crus unobstructed. The first connecting band 21 and the third connecting band 31 are detachably connected by a hook-and-loop fastener or a buckle. The second connecting band 22 and the fourth connecting band 32 are detachably connected by a hook-and-loop fastener or a buckle. The length of the first connecting band 21 is greater than the length of the second connecting band 22, and the length of the third connecting band 31 is greater than the length of the fourth connecting band 32, so that the shorter second connecting band and the shorter fourth connecting band can better fit the user's crus, and the longer first connecting band and the longer third connecting band can better fit the user's thigh. Specifically, the first connecting part 2 is provided on a left side of the knee brace main body 1, and the second connecting part 3 is provided on a right side of the knee brace main body 1. An upper side edge of the knee brace main body is arc-shaped, and a lower side edge of the knee brace main body is arc-shaped. Since the upper side edge of the knee brace main body is arc-shaped, and the lower side edge of the knee brace main body is arc-shaped, when the knee brace main body surrounds and forms the wrapping space, the upper side edge and the lower side edge of the knee brace main body are smoother, which can effectively prevent the upper side edge and the lower side edge of the knee brace main body from curling.

In this embodiment, the knee brace further includes a knee brace cover 9. The knee brace cover 9 is detachably connected to the knee brace main body 1. Through the above structure, the knee brace cover 9 can further protect the knee. For example, when the user falls, the knee brace cover 9 can protect the knee to avoid direct impact on the knee.

In this embodiment, a hardness of the knee brace cover 9 is greater than a hardness of the knee brace main body 1. The knee brace cover 9 includes a plastic cover 91 and a flexible layer 92. The plastic cover 91 is connected to the flexible layer 92, and the flexible layer 92 is detachably connected to the knee brace main body 1. The flexible layer 92 is a flexible cotton layer. The third connecting part 13 is positioned in a center of the knee brace main body 1. The knee brace cover 9 detachably covers the third connecting part 13. The third connecting part 13 is in an annular shape, and the third connecting part 13 protrudes from a surface of the knee brace main body 1. An avoidance through hole 131 is defined in a middle portion of the third connecting part 13. Through the above structure, the arrangement of the knee brace cover 9 and the third connecting part 13 is effectively achieved. The avoidance through hole 131 can make the knee brace main body 1 more breathable, and the flexible layer 92 of the knee brace cover 9 can play a buffering role. When the user falls, the knee brace cover 9 can protect the knee, and the flexible layer 92 can reduce an impact force, so as to reduce the impact force on the knee and protect the knee. The third connecting part is internally equipped with a flexible filling material 1011. Specifically, the filling material is filling cotton.

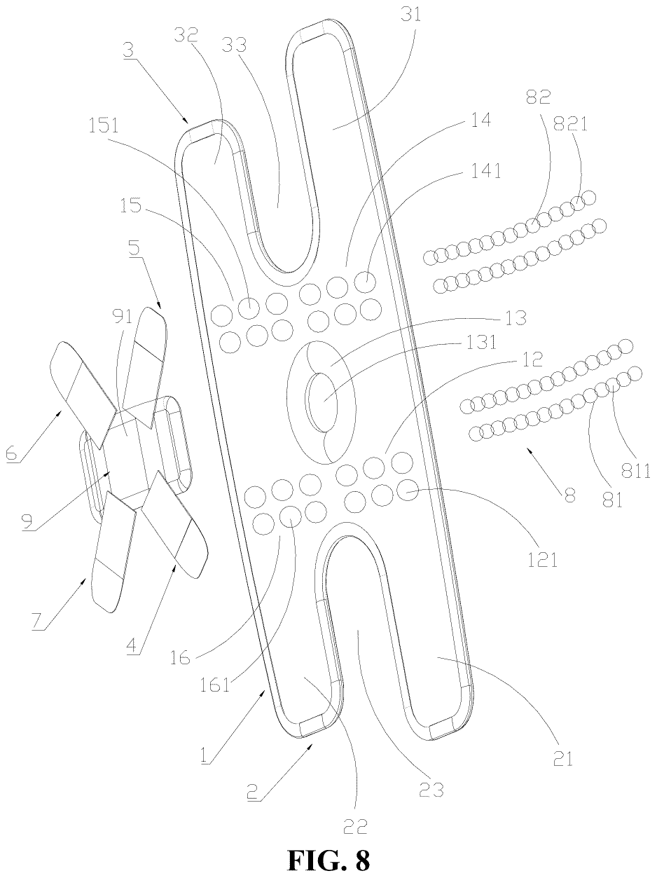

Referring to FIG. 8, in some embodiments, the knee brace further includes a knee brace cover 9. The first end of the first reinforcing band part 4 is detachably connected to the third connecting part 13 through the knee brace cover, and the second end of the first reinforcing band part 4 is detachably connected to the first connecting area 12. The first reinforcing band part 4 is used for tightening the first connecting area 12 in the direction towards the third connecting part 13 and the knee brace cover, so that the first reinforcing band part 4 shrinks the wrapping space 11. Through the above structure, the connection between the first reinforcing band part and the third connecting part has been effectively achieved. The first reinforcing band is connected to the knee brace cover, and the knee brace cover is detachably connected to the third connecting part through a hook-and-loop fastener to attach the first reinforcing band part to the third connecting part.

The above description only describes embodiments of the present disclosure, and is not intended to limit the present disclosure; various modifications and changes can be made to the present disclosure. Any modifications, equivalent substitutions, and improvements made within the spirit and scope of the present disclosure are intended to be included within the scope of the present disclosure.

What is claimed is:

1. A knee brace, comprising:
a knee brace main body, wherein a side wall of the knee brace main body surrounds and forms a wrapping space for wrapping a knee; and
a first reinforcing band part, wherein a first end of the first reinforcing band part is connected to a first part of the knee brace main body, a second end of the first reinforcing band part is detachably connected to a second part of the knee brace main body, and the first reinforcing band part is used for shrinking the wrapping space.

2. The knee brace according to claim 1, wherein the knee brace main body comprises a first connecting area and a third connecting part, the first end of the first reinforcing band part is connected to the third connecting part, the second end of the first reinforcing band part is detachably connected to the first connecting area, and the first reinforcing band part is used for tightening the first connecting area in a direction towards the third connecting part, so that the first reinforcing band part shrinks the wrapping space.

3. The knee brace according to claim 2, wherein the first connecting area is provided with a plurality of first connecting portions, the first reinforcing band part is provided with first connecting fitting portions, and the first connecting fitting portion is selectively connected to one of the plurality of first connecting portions to adjust a size of the wrapping space.

4. The knee brace according to claim 2, further comprising a second reinforcing band part, wherein the knee brace main body further comprises a second connecting area, a first end of the second reinforcing band part is connected to the third connecting part, a second end of the second reinforcing band part is detachably connected to the second connecting area, and the second reinforcing band part is used for tightening the second connecting area in the direction towards the third connecting part, so that the second reinforcing band part shrinks the wrapping space.

5. The knee brace according to claim 4, wherein the second connecting area is provided with a plurality of second connecting portions, the second reinforcing band part is provided with second connecting fitting portions, and the second connecting fitting portion is selectively connected to one of the plurality of second connecting portions to adjust a size of the wrapping space.

6. The knee brace according to claim 4, further comprising a third reinforcing band part, wherein the knee brace main body further comprises a third connecting area, a first end of the third reinforcing band part is connected to the third connecting part, a second end of the third reinforcing band part is detachably connected to the third connecting area, and the third reinforcing band part is used for tightening the third connecting area in the direction towards the third connecting part, so that the third reinforcing band part shrinks the wrapping space.

7. The knee brace according to claim 6, wherein the third connecting area is provided with a plurality of third connecting portions, the third reinforcing band part is provided with third connecting fitting portions, and the third connecting fitting portion is selectively connected to one of the plurality of third connecting portions to adjust a size of the wrapping space.

8. The knee brace according to claim 6, further comprising a fourth reinforcing band part, wherein the knee brace main body further comprises a fourth connecting area, a first end of the fourth reinforcing band part is connected to the third connecting part, a second end of the fourth reinforcing band part is detachably connected to the fourth connecting area, and the fourth reinforcing band part is used for tightening the fourth connecting area in the direction towards the third connecting part, so that the fourth reinforcing band part shrinks the wrapping space.

9. The knee brace according to claim 8, wherein the fourth connecting area is provided with a plurality of fourth connecting portions, the fourth reinforcing band part is provided with fourth connecting fitting portions, and the fourth connecting fitting portion is selectively connected to one of the plurality of fourth connecting portions to adjust a size of the wrapping space.

10. The knee brace according to claim 4, wherein an extension direction of the first reinforcing band part is different from an extension direction of the second reinforcing band part.

11. The knee brace according to claim 8, wherein the third reinforcing band part and the first reinforcing band part extend in opposite directions to each other; the fourth reinforcing band part and the second reinforcing band part extend in opposite directions to each other; and the first reinforcing band part, the second reinforcing band part, the third reinforcing band part, and the fourth reinforcing band part are distributed in a cross shape.

12. The knee brace according to claim 3, wherein the first connecting portion is a first hook-and-loop fitting band, and the first connecting fitting portion is a first hook-and-loop band.

13. The knee brace according to claim 1, wherein one side of the knee brace main body is equipped with a first connecting part, an opposite side of the knee brace main body is equipped with a second connecting part, and the first connecting part is connected to the second connecting part, so that the side wall of the knee brace main body surrounds and forms the wrapping space for wrapping the knee.

14. The knee brace according to claim 13, further comprising a supporting part, wherein the first connecting part is positioned on a left side of the knee brace main body, the second connecting part is positioned on a right side of the knee brace main body, and the supporting part extends from an upper side of the knee brace main body to a lower side of the knee brace main body.

15. The knee brace according to claim 14, wherein the supporting part comprises a plurality of elastic first supporting steel ribs and a plurality of elastic second supporting steel ribs, the plurality of first supporting steel ribs are arranged at intervals on the left side of the knee brace main body, and the plurality of second supporting steel ribs are arranged at intervals on the right side of the knee brace main body.

16. The knee brace according to claim 15, wherein the first supporting steel rib is formed by a plurality of first steel rings being sequentially connected; the second supporting steel rib is formed by a plurality of second steel rings being sequentially connected; a plurality of first installation chambers are defined in the left side of the knee brace main body, a plurality of second installation chambers are defined in the right side of the knee brace main body, the plurality of first installation chambers are separated from each other, the plurality of second installation chambers are separated from each other, the first supporting steel rib is positioned inside the first installation chamber, and the second supporting steel rib is positioned inside the second installation chamber.

17. The knee brace according to claim 13, wherein the first connecting part comprises a protruding first connecting band and a protruding second connecting band, the second connecting part comprises a protruding third connecting band and a protruding fourth connecting band, the first connecting band is detachably connected to the third connecting band, and the second connecting band is detachably connected to the fourth connecting band; a first avoidance gap is defined between the first connecting band and the second connecting band, and a second avoidance gap is defined between the third connecting band and the fourth connecting band; a length of the first connecting band is greater than a length of the second connecting band, and a length of the third connecting band is greater than a length of the fourth connecting band; the first connecting part is provided on a left side of the knee brace main body, the second connecting part is provided on a right side of the knee brace main body, an upper side edge of the knee brace main body is arc-shaped, and a lower side edge of the knee brace main body is arc-shaped.

18. The knee brace according to claim 2, further comprising a knee brace cover, wherein the knee brace cover is detachably connected to the knee brace main body.

19. The knee brace according to claim 18, wherein a hardness of the knee brace cover is greater than a hardness of the knee brace main body; the knee brace cover comprises a plastic cover and a flexible layer, the plastic cover is connected to the flexible layer, and the flexible layer is detachably connected to the knee brace main body; the third connecting part is positioned in a center of the knee brace main body; the knee brace cover detachably covers the third connecting part; the third connecting part is in an annular shape, the third connecting part protrudes from a surface of the knee brace main body, and an avoidance through hole is defined in a middle portion of the third connecting part.

20. The knee brace according to claim 2, further comprising a knee brace cover, wherein the first end of the first reinforcing band part is detachably connected to the third connecting part through the knee brace cover, the second end of the first reinforcing band part is detachably connected to the first connecting area, and the first reinforcing band part is used for tightening the first connecting area in the direction towards the third connecting part and the knee brace cover, so that the first reinforcing band part shrinks the wrapping space.
